(19) United States
(12) Patent Application Publication
(10) Pub. No.: US 2017/0333260 A1
(43) Pub. Date: Nov. 23, 2017

(54) SANITARY NAPKIN AND ASSOCIATED USE THEREOF

(71) Applicant: Gina Garner, Jacksonville, FL (US)

(72) Inventor: Gina Garner, Jacksonville, FL (US)

(21) Appl. No.: 15/157,239

(22) Filed: May 17, 2016

(51) Int. Cl. A61F 13/475; A61F 13/472; A61F 13/47; A61F 13/15

(52) U.S. Cl. CPC A61F 13/4757; A61F 13/47218; A61F 2013/4708; A61F 2013/16

(57) ABSTRACT

A sanitary napkin increases and ensures secure protection with an adhesive having a solid alignment lock. The sanitary napkin preferably has an extended length and is provided with a sponge-like rear filter to absorb tunnel-patterned bodily excretions. A generally diamond shaped padding absorbs bodily excretions from the vaginal region. Notably, the stronger adhesive locks the body in alignment with an undergarment.

### CROSS REFERENCE TO RELATED APPLICATIONS

Not Applicable.

### STATEMENT REGARDING FEDERALLY SPONSORED RESEARCH OR DEVELOPMENT

Not Applicable.

### REFERENCE TO A MICROFICHE APPENDIX

Not Applicable.

### BACKGROUND OF THE DISCLOSURE

### Technical Field

Exemplary embodiment(s) of the present disclosure relate to sanitary napkins and, more particularly, to a sanitary napkin for preventing vaginal leakage during heavy menstrual excretions.

### Prior Art

Absorbent personal care articles intended to absorb discharged bodily fluids are well known in the art. Such absorbent articles generally comprise a fibrous mass or other absorbent core which can absorb and hold body fluids. Similarly, it is well known that feminine care articles have been employed to absorb and hold liquids, such as urine and/or menses. A typical structure of an absorbent article includes a fluid impermeable back sheet, a fluid permeable top sheet and an absorbent core positioned between the back sheet and the top sheet. Prior absorbent articles have also included various other features to improve fluid handling, such as intake layers, distribution layers, retention layers and the like. In these absorbent personal care articles, the top sheet is the body-facing side of the absorbent article and the back sheet is the garment-facing side of the absorbent article.

Generally, the absorbent articles are held in place during use by using the wearer's waist and elastic materials in the waist portion of the absorbent product in place during use, in the case of pant-like garments, such as diapers and training pants, or by attaching the absorbent article to the underwear or undergarment of a user, in the case of pads or liners. Current methods of attaching the absorbent article to the underwear or undergarment of a user include placing an adhesive on the garment-facing side of the back sheet, having optional flaps (wings) that extend from the longitudinal sides of the absorbent article which wrap around the crotch portion of the underwear or undergarment of the user and a combination of the adhesive and the flaps.

Similarly, absorbent articles that are attached to the underwear or undergarment of a user can also be uncomfortable for the user. This is because during normal movement of the body, portions of the body place opposed forces on the undergarment, which may cause the undergarment to be bunched or twisted. When this occurs, any absorbent article attached to the underwear or undergarment may also become bunched or twisted, causing discomfort to the wearer of the absorbent article. For example, the presence and absence of pressure from the absorbent article on the inner thighs as the user moves, which is often described by users as feeling “like a diaper”, is one source which compromises comfort for users of conventional absorbent articles, including liners, ultra-thin absorbent pads and maxi pads. In addition, the movement of the user or deformation of the underwear while being worn may also cause the absorbent article to have a poor fit against the body of the user, which could result in leaks from the absorbent article.

Another disadvantage of conventional absorbent articles is that the silhouette or outline of the absorbent article may be visible to others through the clothing of the user. Even currently available ultra-thin absorbent articles may be visible through tight fitting outer clothing of a user. Therefore, conventional absorbent personal care articles do not always provide discretion for users.

Furthermore, while I have never experienced excess excretions to the extreme of wearing a tampon in combination with a sanitary napkin 10, unfortunately, I have family and friends that did. We need something secure enough especially after childbirth, surgical procedures, and overall just having heavy bodily excretions during the menstrual cycle.

Accordingly, a need remains for a sanitary napkin in order to overcome at least one prior art shortcoming. The exemplary embodiment(s) satisfy such a need by providing a sanitary napkin that is convenient and easy to use, lightweight yet durable in design, versatile in its applications, and designed for preventing vaginal leakage during heavy menstrual excretions.

### BRIEF DESCRIPTION OF THE SEVERAL VIEWS OF THE DRAWINGS

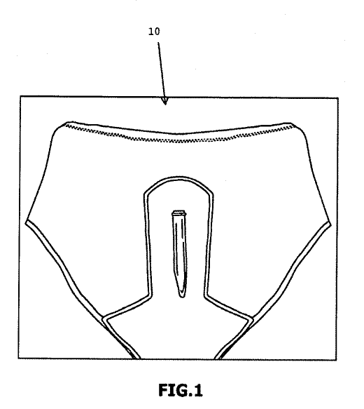

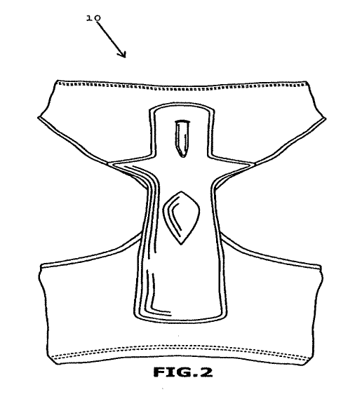

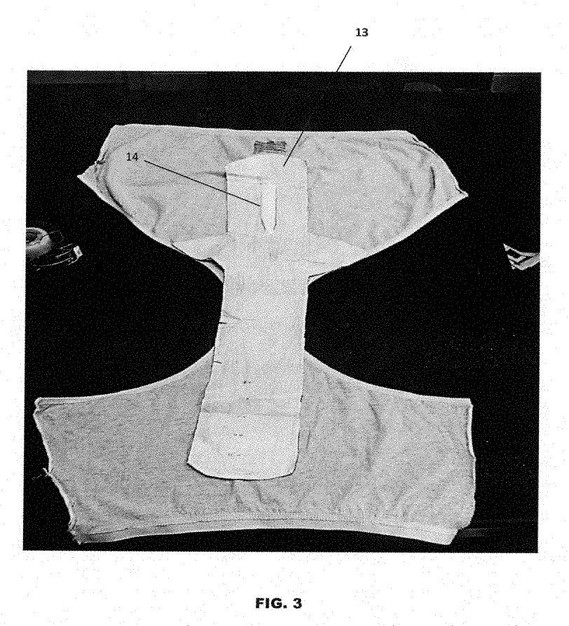

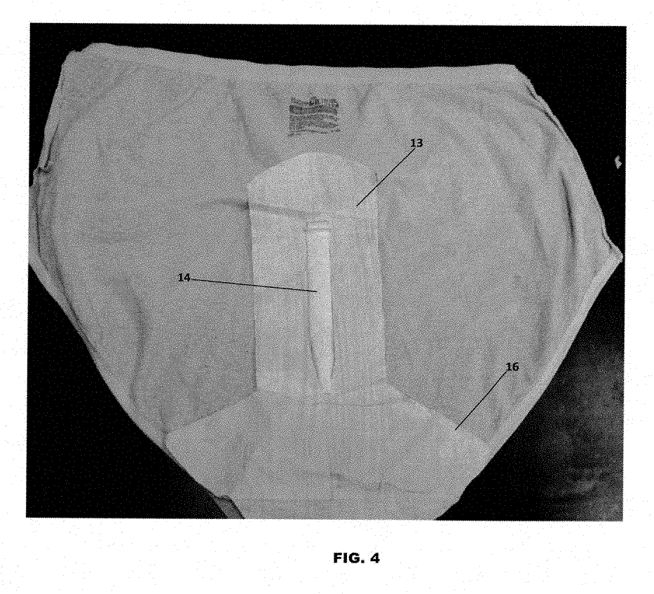

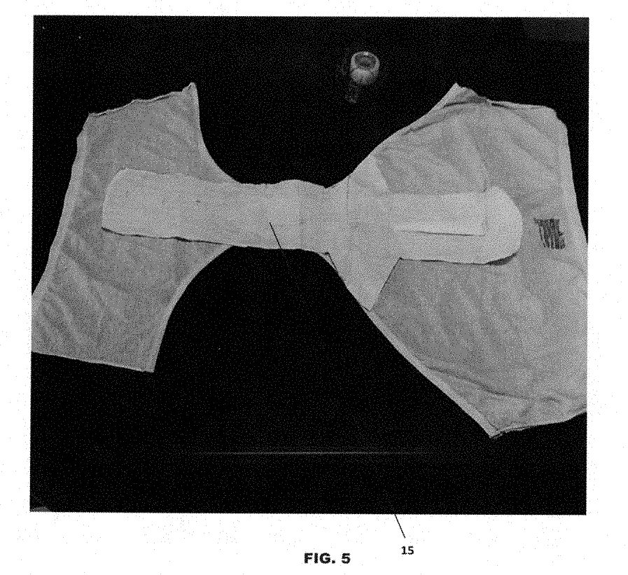

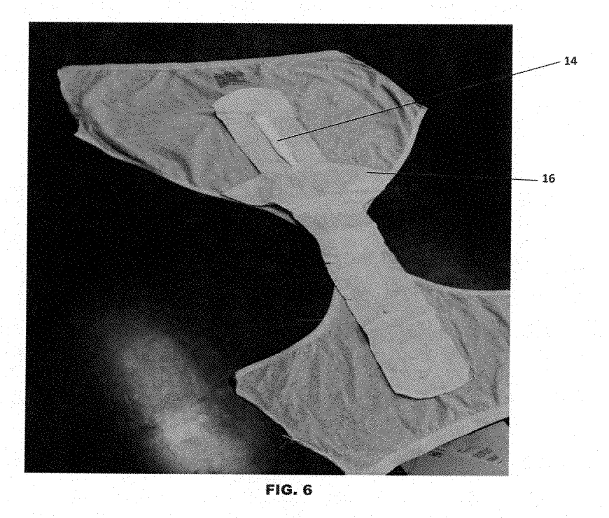

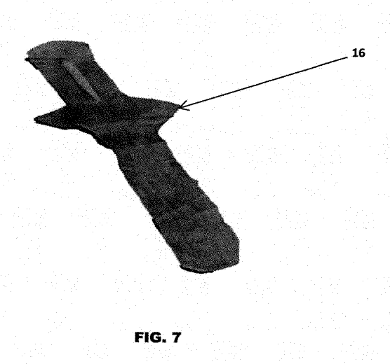

The novel features believed to be characteristic of the exemplary embodiment(s) are set forth with particularity in the appended claims. The disclosure itself, however, both as to its organization and method of operation, together with further objects and advantages thereof, may best be understood by reference to the following description taken in connection with the accompanying drawings in which: FIG. 1, FIG. 2, FIG. 3, FIG. 4, FIG. 5, FIG. 6, & FIG. 7.

The figures generally include various perspective/elevational view(s) showing a sanitary napkin for preventing vaginal leakage during heavy menstrual excretions, in accordance with non-limiting exemplary embodiment(s) of the present disclosure.

### DETAILED DESCRIPTION OF THE DISCLOSURE

The non-limiting exemplary embodiment(s) will now be described more fully hereinafter with reference to the accompanying drawings, in which a preferred embodiment of the disclosure is shown. Such exemplary embodiment(s) may, however, be embodied in many different forms and should not be construed as limited to the embodiment set forth herein. Rather, these embodiment(s) are provided so that this application will be thorough and complete, and will fully convey the true scope of the disclosure to those skilled in the art.

The below disclosed subject matter is to be considered illustrative, and not restrictive, and any appended claim(s) are intended to cover all such modifications, enhancements, and other embodiment(s) which fall within the true scope of the non-limiting exemplary embodiment(s). Thus, to the maximum extent allowed by law, the scope of the non-limiting exemplary embodiment(s) is to be determined by the broadest permissible interpretation of the claim(s) and their equivalents, and shall not be restricted or limited by the foregoing detailed description.

References in the specification to “an exemplary embodiment”, “an embodiment”, “a preferred embodiment”, “an alternative embodiment” and similar phrases mean that a particular feature, structure, or characteristic described in connection with the embodiment(s) is included in at least an embodiment of the disclosure. The appearances of the phrase “a nonlimiting exemplary embodiment” in various places in the specification are not necessarily all meant to refer to the same embodiment.

A non-limiting exemplary embodiment of the present disclosure is referred to generally in the figures and is intended to provide a sanitary napkin 10 for preventing vaginal leakage during heavy menstrual excretions. It should be understood that the exemplary embodiment may be used to absorb many different types of leakage, and should not be limited to any particular vaginal leakage described herein.

Referring to the figures in general, in a non-limiting exemplary embodiment, the sanitary napkin 10 increases and ensures secure protection with an adhesive having a solid alignment lock. The sanitary napkin 10 preferably has an extended length and is provided with a sponge-like rear filter 14 to absorb tunnel-patterned bodily excretions. A generally diamond shaped central padding 15 absorbs bodily excretions from the vaginal region. Notably, the stronger adhesive locks the body 13 in alignment with an undergarment 20 or underwear. A generally triangular head shaped side padding 16 supports 14 for the pelvic overflow secretions.

In a non-limiting exemplary embodiment, the shape and size of the sanitary napkin 10 may be molded in a suitable manner to promote proper body posture.

In a non-limiting exemplary embodiment, conventional take-away wings can be replaced with the generally diamond shape central padding 15 and generally triangular head shaped side padding 16 that is attached to the body 13 and thereby easily fits in the undergarment 20. In this manner, the sanitary napkin 10 is secure enough to reduce the need to wear a tampon in addition to wearing a sanitary napkin 10.

In a non-limiting exemplary embodiment, the body 13 may be 22 to 25 inches long. The rear of the body 13 may be approximately 4 inches wide. The middle of the body 13 may be 3 to 3.5 inches wide and the front of the body 13 may be 3 inches wide. About 2.5 inches from the rear of body 13 is a 3 inch length, 0.5 inch wide sponge-like rear filter 14 that blocks and catches unsecure runaway excretions, which creep on clothes in a tunnel pattern while sitting up or lying down. Such a rear filter 14 may have a generally rectilinear shape and extend generally parallel to a longitudinal length of the body 13. The rear filter 14 may have a cylindrical outer surface registered proximal to the rear end of the body 13.

In a non-limiting exemplary embodiment, a user may be sitting for hours at a time, or take a nap, and upon getting up she may find that a runaway tunnel of bodily excretions, which did not meet the pad, are on her clothing. Even if you have on an extra-long overnight pad, the excretions may get on your clothes and not the pad. I've found myself often putting two pads together just for the security of such tunnel excretions. This is where the 22 to 25 inches of the body 13 comes in handy for the front to back security and away with embarrassing moments and messy clean ups after failed security of a conventional sanitary napkin 10. This multi-functional sanitary napkin 10 is not only for night time wear but also for daytime as well.

In a non-limiting exemplary embodiment, the vaginal central padding 15 of body 13 may have an extra secure lifted layer to securely lock in odor and wetness. About 6.5 inches from the rear of the body 13 may be a half-diamond shape cut out padding 15 to also ensure lower pelvic security. The central padding 15 and 16 has an absorbent layer to remind you the quality of protection is concealed.

In a non-limiting exemplary embodiment, the quantity of the sanitary napkins 10 may possibly include 24 per package. Possibly 8 with or without the diamond padding 15 and/or rear filter 14. Possibly 8 with the diamond padding 15 and/or rear filter 14. For individuals with increased weight and size, there may be larger product to accommodate for the proper capacity size.

There has thus been outlined, rather broadly, the more important features of the disclosure in order that the detailed description thereof that follows may be better understood, and in order that the present contribution to the art may be better appreciated.

It is noted the purpose of the foregoing abstract is to enable the U.S. Patent and Trademark Office and the public generally, especially the scientists, engineers and practitioners in the art who are not familiar with patent or legal terms or phraseology, to determine quickly from a cursory inspection the nature and essence of the technical disclosure of the application. The abstract is neither intended to define the disclosure of the application, nor is it intended to be limiting as to the scope of the disclosure in any way.

While the disclosure has been described with respect to certain specific embodiment(s), it will be appreciated that many modifications and changes may be made by those skilled in the art without departing from the spirit of the disclosure. It is intended, therefore, by the description hereinabove to cover all such modifications and changes as fall within the true spirit and scope of the disclosure. In particular, with respect to the above description, it is to be realized that the optimum dimensional relationships for the parts of the exemplary embodiment(s) may include variations in size, materials, shape, form, function and manner of operation.

What is claimed as new and what is desired to secure by Letters Patent of the United States is:

1. A sanitary napkin for preventing vaginal leakage during heavy menstrual excretions, said sanitary napkin comprising:
a body
a central padding attached to a middle region of said body; and
a rear filter suitably sized and shaped to absorb bodily excretions that pass beyond the central absorption pad.
